(19) United States
(12) Patent Application Publication
(10) Pub. No.: US 2023/0064454 A1
(43) Pub. Date: Mar. 2, 2023

(54) SYSTEM AND METHOD FOR GENERATING SOIL MOISTURE DATA FROM SATELLITE IMAGERY USING DEEP LEARNING MODEL

(71) Applicant: Satsure Analytics India Private Limited, Bangalore (IN)

(72) Inventors: Catherin R Sebastian, Bangalore (IN); Sanjutha Indrajit, Bangalore (IN); Pradeep Kumar Bisen, Bangalore (IN)

(21) Appl. No.: 17/900,711

(22) Filed: Aug. 31, 2022

(30) Foreign Application Priority Data
Aug. 31, 2021 (IN) 202141039333

(51) Int. Cl. G01N 33/24; G06T 3/40; G06T 5/00; G06T 7/00; G06T 5/50; G01S 13/90; G01N 22/04

(52) U.S. Cl. CPC G01N 33/246; G06T 3/4007; G06T 5/002; G06T 5/006; G06T 7/97; G06T 5/50; G01S 13/9021; G01N 22/04; G06T 2207/10036; G06T 2207/10044; G06T 2207/20192; G06T 2207/20221; G06T 2207/20081

(57) ABSTRACT

A system and method for generating soil moisture data from satellite images of a geographical area using a deep learning model 108 is provided. The system includes one or more satellites 102A-C, a soil moisture data generator server 106. The method includes, (i) receiving, by a soil moisture data generator server, satellite images of the geographical area, (ii) pre-processing first set of satellite images, second set of satellite images, and third set of satellite images, (iii) interpolating, using spline interpolation, pre-processed first set of images, pre-processed second set of images, and pre-processed third set of images to generate high-resolution set of images, (iv) generating hydrological parameters from the high-resolution set of images, (v) training, a deep learning model, by providing historical hydrological parameters and historical soil moisture data associated with historical satellite images as training data to generate trained deep learning model, (v) generating soil moisture data on daily basis.

### CROSS-REFERENCE TO PRIOR-FILED PATENT APPLICATIONS

This application claims priority from the Indian provisional application no. 202141039333 filed on Aug. 31, 2021, which is herein incorporated by reference.

### TECHNICAL FIELD

The embodiments herein generally relate to satellite image processing, more particularly, a system and method for generating soil moisture data from satellite images of a geographical area using a deep learning model on a daily basis.

### DESCRIPTION OF THE RELATED ART

Soil moisture is the total amount of water, including, water vapor, in unsaturated soil. Soil moisture represents water that resides in the pores of the soil. The level of soil moisture is determined by a host of factors beyond weather conditions, including soil type, and associated vegetation. The amount of soil moisture can have significantly different implications depending on location, season, soil type, and depth. Soil moisture data is majorly utilized in agricultural monitoring, drought, and flood forecasting, forest fire prediction, water supply management, and other natural resource activities.

In some existing techniques, soil moisture data is obtained by optical, passive, and active microwave sensors. Optical sensors may not retrieve data if it is cloudy during the satellite pass and hence cannot provide the retrieval data throughout the year. The optical/thermal data fails to control cloud and aerosol issues in soil moisture data. Microwave remote sensing enables the measurement of surface soil moisture for different conditions of vegetable cover, roughness, rainfall, and anthropogenic activities within reasonable error bounds. Hence, microwave remote sensing of soil moisture is advantageous over other spectral regions: (1) the microwave emissions are unaffected by clouds and aerosols, providing all-weather coverage; (2) the effect of vegetation is not that significant, allowing the observation of underlying surfaces; (3) the measurement is based on the dielectric properties of the material, which for soil is a function of the amount of moisture present.

Therefore, there arises a need to address the aforementioned technical drawbacks in existing technologies in generating soil moisture data.

### SUMMARY

In view of the foregoing, an embodiment herein provides a system for generating soil moisture data from a plurality of satellite images of a geographical area using a deep learning model for managing irrigation in the geographical area. The system includes satellites that capture satellite images of the geographical area. The satellite images include a set of spectral bands. The system includes a soil moisture data generator server that receives the satellite images of the geographical area from the satellites. The satellite images include a first set of satellite images that are captured in a first set of spectral bands by a first satellite, a second set of satellite images that are captured in a second set of spectral bands by a second satellite and a third set of satellite images that are captured in a third set of spectral bands by a third satellite. The soil moisture data generator server includes a memory that stores a database, and a processor in communication with the memory, the processor is configured to (i) generate a pre-processed first set of images, a pre-processed second set of images, and a pre-processed third set of images by pre-processing the first set of satellite images, the second set of satellite images, and the third set of satellite images; (ii) interpolate the pre-processed first set of images, the pre-processed second set of images, and the pre-processed third set of images to generate a high-resolution set of images, the high-resolution set of images are generated by increasing a temporal resolution of the pre-processed first set of images, the pre-processed second set of images, and the pre-processed third set of images to a daily scale using a spline interpolation; (iii) generate hydrological parameters from the high-resolution set of images; (iv) train a deep learning model by providing historical hydrological parameters and historical soil moisture data associated with historical satellite images as training data to generate a trained deep learning model; and (v) generate, using the trained deep learning model, high resolution volumetric soil moisture data of the geographical area on daily basis by relating a microwave polarization index to soil moisture data estimates, thereby managing the irrigation in the geographical area using the high resolution volumetric soil moisture data.

In some embodiments, the processor is configured to pre-process the first set of satellite images, and the second set of satellite images by, (i) applying atmospheric corrections to remove effects of atmosphere on the first set of satellite images to obtain the atmospherically corrected first set of satellite images and the atmospherically corrected second set of satellite images, (ii) applying a border noise removal on the atmospherically corrected first set of images to remove low-intensity noise and invalid data on edges of the atmospherically corrected first set of images, (iii) calibrating the atmospherically corrected second set of satellite images to convert digital pixel values of the atmospherically corrected first set of images into radiometrically calibrated synthetic aperture radar (SAR) backscatter data, (iv) applying a terrain correction on the SAR backscatter data by removing distortions that are related to a side geometry, and (v) converting the SAR backscatter data into the top of atmosphere reflectance.

In some embodiments, the processor is configured to apply a darkest pixel correction that corresponds to a large water body to the second set of satellite images. In some embodiments, the processor is configured to divide the geographical area into grids by regularizing the third set of satellite images. In some embodiments, the processor is configured to apply an aerosol correction to the second set of images to eliminate atmospheric effects on the top of atmosphere reflectance. In some embodiments, the processor is configured to compute a normalized difference vegetation index (NDVI) and land surface temperature from the second set of satellite images.

In some embodiments, the processor is configured to train the deep learning model by, (i) extracting a first set of features from the historical satellite images, (ii) merging the first set of features to obtain feature maps, the feature maps include at least one of corners, or edges in the historical satellite images, (iii) extracting a second set of features by decreasing a size of each feature map independently, (iv) determining the set of historical hydrological parameters from the second set of features using a max-pooling method, (v) backpropagating a loss function to the deep learning model to optimize the se of historical hydrological parameters and the second set of features such that the loss function becomes zero, (vi) generating an optimized set of historical hydrological parameters as the historical hydrological parameters of the historical satellite images, and (vii) providing the historical hydrological parameters as the training data for training the deep learning model.

In some embodiments, the feature maps are generated using a convolution filter to the one or more historical satellite images.

In some embodiments, a maximum element of a region of each feature map is selected to determine the set of historical hydrological parameters from the second set of features using the max pooling method.

In some embodiments, the processor is configured to analyze a vegetation-based lookup table to relate the microwave polarization index to the soil moisture data estimates using a soil moisture data upscaling algorithm, a volumetric heat capacity of soil increases when a soil layer becomes wetter and a greater water content corresponds to a smaller temperature variation.

In one aspect, there is provided one or more non-transitory computer-readable storage mediums storing one or more sequences of instructions, which when executed by one or more processors, causes a method for generating soil moisture data from satellite images of a geographical area using a deep learning model for managing irrigation in the geographical area is provided. The method includes receiving, by a soil moisture data generator server, satellite images of the geographical area from satellites. The satellite images include a first set of satellite images that are captured in a first set of spectral bands by a first satellite, a second set of satellite images that are captured in a second set of spectral bands by a second satellite, and a third se of satellite images that are captured in a third set of spectral bands by a third satellite. The method includes generating a pre-processed first set of images, a pre-processed second se of images, and a pre-processed third se of images by pre-processing the first set of satellite images, the second set of satellite images, and the third se of satellite images, The method includes interpolating the pre-processed first set of images, the pre-processed second set of images, and the pre-processed third set of images to generate a high-resolution set of images, the high-resolution set of images are generated by increasing a temporal resolution of the pre-processed first set of images, the pre-processed second set of images, and the pre-processed third set of images to a daily scale using a spline interpolation. The method includes generating hydrological parameters from the high-resolution set of images. The method includes training a deep teaming model by providing historical hydrological parameters and historical soil moisture data associated with historical satellite images as training data to generate a trained deep learning model. The method includes generating, using the trained deep learning model, high resolution volumetric soil moisture data of the geographical area on daily basis by relating a microwave polarization index to soil moisture data estimates, thereby managing the irrigation in the geographical area using the high resolution volumetric soil moisture data.

In another aspect, a processor-implemented method for generating soil moisture data from satellite images of a geographical area using a deep learning model for managing irrigation in the geographical area is provided. The method includes receiving, by a soil moisture data generator server, satellite images of the geographical area from satellites. The satellite images include a first set of satellite images that are captured in a first set of spectral bands by a first satellite, a second set of satellite images that are captured in a second set of spectral bands by a second satellite, and a third set of satellite images that are captured in a third set of spectral bands by a third satellite. The method includes generating a pre-processed first set of images, a pre-processed second set of images, and a pre-processed third set of images by pre-processing the first set of satellite images, the second set of satellite images, and the third set of satellite images. The method includes interpolating the pre-processed first set of images, the pre-processed second set of images, and the pre-processed third set of images to generate a high-resolution set of images, the high-resolution set of images are generated by increasing a temporal resolution of the pre-processed first set of images, the pre-processed second set of images, and the pre-processed third set of images to a daily scale using a spline interpolation. The method includes generating hydrological parameters from the high-resolution set of images. The method includes training a deep learning model by providing historical hydrological parameters and historical soil moisture data associated with historical satellite images as training data to generate a trained deep learning model. The method includes generating, using the trained deep learning model, high resolution volumetric soil moisture data of the geographical area on daily basis by relating a microwave polarization index to soil moisture data estimates, thereby managing the irrigation in the geographical area using the high resolution volumetric soil moisture data.

In some embodiments, the method includes pre-processing of the first set of satellite images, and the second set of satellite images by, (i) applying atmospheric corrections to remove effects of atmosphere on the first set of satellite images to obtain the atmospherically corrected first set of satellite images and the atmospherically corrected second set of satellite images, (ii) applying a border noise removal on the atmospherically corrected first set of images to remove low-intensity noise and invalid data on edges of the atmospherically corrected first set of images, (iii) calibrating the atmospherically corrected second set of satellite images to convert digital pixel values of the atmospherically corrected first set of images into radiometrically calibrated synthetic aperture radar (SAR) backscatter data, (iv) applying a terrain correction on the SAR backscatter data by removing distortions that are related to a side geometry, and (v) converting the SAR backscatter data into the top of atmosphere reflectance.

In some embodiments, the method includes training the deep learning model by, (i) extracting a first set of features from the historical satellite images, (ii) merging the first set of features to obtain feature maps, the feature maps include at least one of corners, or edges in the historical satellite images, (iii) extracting a second set of features by decreasing a size of each feature map independently, (iv) determining the set of historical hydrological parameters from the second set of features using a max-pooling method, (v) backpropagating a loss function to the deep learning model to optimize the set of historical hydrological parameters and the second set of features such that the loss function becomes zero, (vi) generating an optimized set of historical hydrological parameters as the historical hydrological parameters of the historical satellite images, and (vii) providing the historical hydrological parameters as the training data for training the deep learning model.

In some embodiments, the method includes applying a darkest pixel correction that corresponds to a large water body to the second set of satellite images.

In some embodiments, the method includes dividing the geographical area into grids by regularizing the third set of satellite images.

In some embodiments, the method includes applying an aerosol correction to the second set of images to eliminate atmospheric effects on the top of atmosphere reflectance. In some embodiments, the method includes computing a normalized difference vegetation index (NDVI) and land surface temperature from the second set of satellite images.

In some embodiments, the feature maps are generated using a convolution filter to the one or more historical satellite images.

In some embodiments, a maximum element of a region of each feature map is selected to determine the set of historical hydrological parameters from the second se of features using the max pooling method.

In some embodiments, the processor is configured to analyze a vegetation-based lookup table to relate the microwave polarization index to the soil moisture data estimates using a soil moisture data upscaling algorithm, a volumetric heat capacity of soil increases when a soil layer becomes wetter and a greater water content corresponds to a smaller temperature variation.

The system and method of generating soil moisture data from satellite imagery using a deep learning model are provided. The system provides real-time soil moisture data at 20 meters resolution on a daily basis. Monitoring the rapidly changing risk factors of the agriculture field requires data at a high temporal scale. Most of the farms in developing countries are less than a hectare, so the existing course resolution soil moisture data would not provide beneficial information on in-farm variations. The system provides data at a high-spatiotemporal resolution, which is adequate enough to monitor moisture variations in a small-sized farm. The daily product will allow one to monitor the variations and make necessary changes in cropping patterns or agricultural inputs way before any damage occurs, agriculture and water resource management. The high spatio-temporal resolution analysis based on soil moisture provides detailed information about the changing soil moisture status, indicating when crops are at risk for stress, when to irrigate, and when to stop. By irrigating according to soil moisture readings, it's possible to apply water when it's needed and where it's needed. Therefore, irrigation can be managed precisely. The system predicts landslides, floods, and fires, using the soil moisture data, Soil moisture index calculated based on field capacity and permanent wilting can provide insights on upcoming agricultural drought/flood days before its onset thereby allowing farmers to take necessary actions. As the data is available from 2010, the system can provide a better historic drought assessment. The important and most error-prone element in budgeting water is the water stored in the land system. Surface soil moisture gives the estimate of the amount of moisture stored in the land system after runoff, infiltration, and evapotranspiration losses. The system enables better water budgeting through the soil moisture data. The system ensures crop insurance. Agriculture is exposed to a wide spectrum of risks. With water being the limiting factor in many countries, analysis of soil moisture present in the topsoil is beneficial for insurance providers to predict drought risks.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF DRAWINGS

The embodiments herein will be better understood from the following detailed description with reference to the drawings, in which:

FIG. 1 illustrates a system for generating soil moisture data on daily basis using a deep learning model according to some embodiments herein;

FIG. 2 illustrates a block diagram of a soil moisture data generator server according to some embodiments herein;

FIG. 3 illustrates a block diagram of a pre-processing module according to some embodiments herein;

FIG. 4 illustrates a block diagram of a deep learning model according to some embodiments herein;

FIG. 5 illustrates an exemplary view of spatial variation of soil moisture data according to some embodiments herein;

FIG. 6 illustrates a graphical representation of a temporal plot of soil moisture data according to some embodiments herein;

FIG. 7 is a flow diagram of a method for generating soil moisture data from satellite images of a geographical area using a deep learning model according to some embodiments herein; and

FIG. 8 is a schematic diagram of a computer architecture in accordance with embodiments herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be constructed as limiting the scope of the embodiments herein.

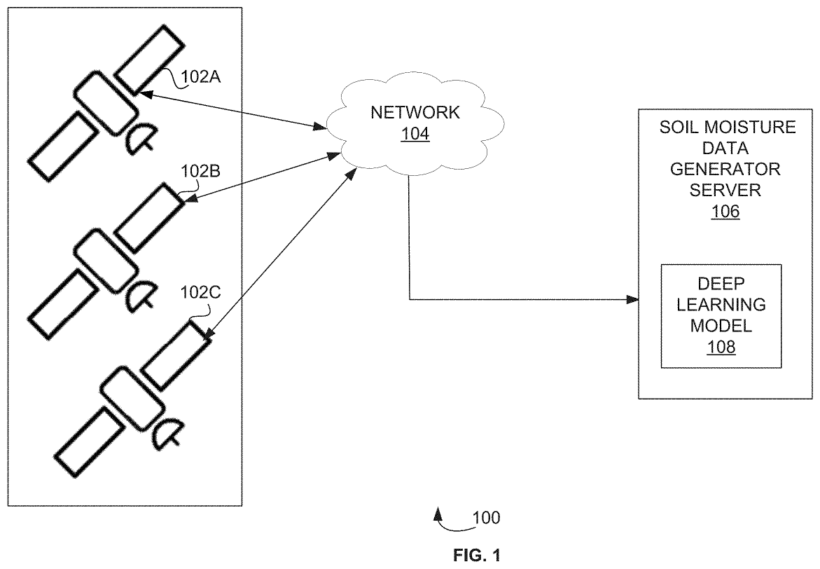

As mentioned, there remains a need for a system and method for generating soil moisture data from satellite images of a geographical area using a deep learning model on daily basis. Referring now to the drawings, and more particularly to FIGS. 1 through 8, where similar reference characters denote corresponding features consistently throughout the figures, and various embodiments are shown.

The following terms are referred to in the description, which is briefly described below:

Sentinel-1 satellites survey every 12 days around the earth and carry a C-band synthetic-aperture radar (SAR) instrument which compares the transmitted and received power to yield a variable called the backscattering coefficient (σₒ), which can be related to the surface reflectivity. The C-SAR instrument provides an all-weather, day and night imaging capability to capture measurement data at high and medium resolutions for land, coastal zones and ice observations. To achieve frequent revisits and high mission availability, two identical Sentinel-1 satellites (Sentinel-1A and Sentinel-1B) operate together. The satellites are phased 180 degrees from each other in the same orbit. This allows for what would be a 12-day revisit cycle to be completed in 6-7 days.

Landsat-8 satellites survey every 16 days around the earth and collect multi-spectral image data affording seasonal coverage of the global landmasses. An operational land imager of Landsat-8 captures images in 9 spectral bands.

Advanced microwave scanning radiometer (AMSR2)—The Advanced Microwave Scanning Radiometer 2 (AMSR2) instrument was launched aboard the Japanese Space Exploration Agency Global Change Observation Mission 1st-Water, “SHIZUKU” (GCOM-W1) satellite on May 18, 2012. AMSR2 provides data on global precipitation, ocean wind speed, water vapor, sea ice concentration, brightness temperature, and soil moisture. The multi-purpose microwave (MW) imager of the AMSR2 provides global coverage once per day.

FIG. 1 illustrates a system 100 for generating soil moisture data from satellite images of a geographical area using a deep learning model 108 on daily basis according to some embodiments herein. The system 100 includes one or more satellites 102A, 102B, and 102C, and a soil moisture data generator server 106 that includes a deep learning model 108. The soil moisture data generator server 106 includes a device processor and a non-transitory computer-readable storage medium storing one or more sequences of instructions, which when executed by the device processor causes the processing of satellite imagery for generating soil moisture data on daily basis. The soil moisture data generator server 106 receives a first set of images, a second set of images, and a third set of images from one or more satellites 102A, 102B, and 102C through a network 104. The network 104 may include, but is not limited to, a wireless network, a wired network, a combination of the wired network and the wireless network or Internet, and the like. The soil moisture data generator server 106 may be a handheld device, a mobile phone, a kindle, a PDA (Personal Digital Assistant), a tablet, a music player, a computer, an onsite/remote server, an electronic notebook or a smartphone.

The first set of images may be from Sentinel-1 satellite 102A. The second set of images may be from a Landsat-8 satellite 102B. The third set of images may be from AMSR2 satellite 102C. The soil moisture data generator server 106 obtain the pre-processed first set of images, the pre-processed second set of images, and the pre-processed third set of images by pre-processing the first set of images, the second set of images, and the third set of images.

The soil moisture data generator server 106 interpolates the pre-processed first set of images, the pre-processed second set of images, and the pre-processed third set of images to generate a high-resolution set of images using spline interpolation. The spline interpolation is used to increase the temporal resolution of the pre-processed images to a daily scale. In spline interpolation, the interpolant is a type of piecewise polynomial called a spline. The soil moisture data generator server 106 interpolates for downscaling input data for finer spatial resolution. The soil moisture data generator server 106 obtains one or more hydrological parameters from the high-resolution set of images. The deep learning model 108 is trained by providing historical hydrological parameters and historical soil moisture data associated with historical satellite images as training data to generate a trained deep learning model. In some embodiments, the deep learning model 108 is trained by an averaging method to downscale descending/ascending passive microwave sensor data. In some embodiments, the deep learning model 108 is trained by images with 100×100-pixel size, an input data, for example, NDVI (normalized difference vegetation index), temperature, and backscatter coefficient with auxiliary soil moisture information (AMSR2) data resampled to 20 meters resolution. The soil moisture data generator server 106 generates high resolution volumetric soil moisture data of the geographical area on daily basis using the trained deep learning model by relating a microwave polarization index to soil moisture data estimates, thereby managing the irrigation in the geographical area using the high resolution volumetric soil moisture data.

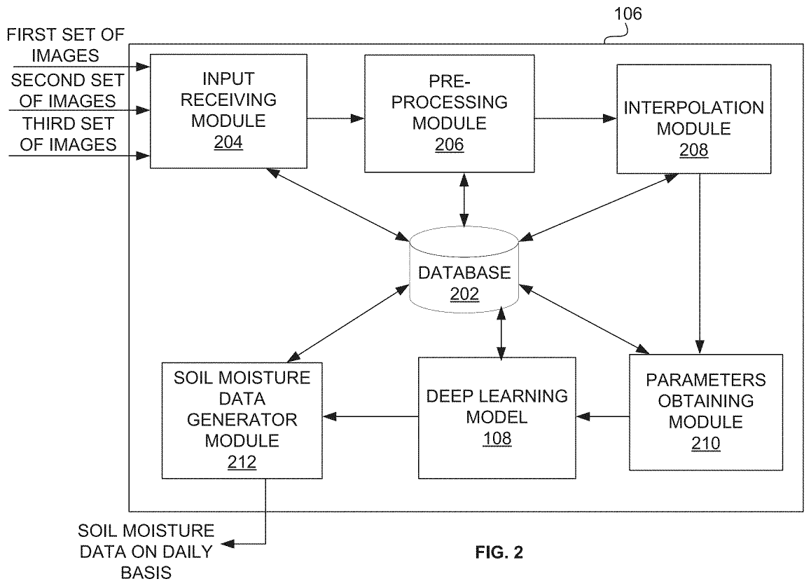

FIG. 2 illustrates a block diagram of a soil moisture data generator server 106 according to some embodiments herein. The block diagram of the soil moisture data generator server 106 includes a database 202, an input receiving module 204, a pre-processing module 206, an interpolation module 208, a parameters obtaining module 210, a deep learning model 108, and a soil moisture data generator module 212. The input receiving module 204 receives a first set of images, a second set of images, and a third set of i mages from one or more satellites 102A, 102B, and 102C. The first set of images, the second set of images, and the third set of images are stored in the database 202. In some embodiments, the first set of images may be from Sentinel-1 satellite 102A, the second set of images may be from Landsat-8 satellite 102B and the third set of images may be from AMSR2 satellite 102C. The pre-processing module 206 selects one or more spectral bands, maybe SAR (Synthetic Aperture Radar), NIR (Near Infrared), SWIR (Short Wave InfraRed), and RGB (Red Green Blue) from the first set of images and the second set of images the one or more satellites 102A and 102B. The pre-processing module 206 obtains the pre-processed first set of images, the pre-processed second set of images, and the pre-processed third set of images by pre-processing the first set of satellite images, the second set of satellite images, and the third set of satellite images.

The interpolation module 208 interpolates the pre-processed first set of images, the pre-processed second set of images, and the pre-processed third set of images to generate a high-resolution set of images. The high-resolution set of images is generated by increasing a temporal resolution of the pre-processed first set of images, the pre-processed second set of images, and the pre-processed third set of images to a daily scale using a spline interpolation. The parameters obtaining module 210 obtain hydrological parameters from the high-resolution set of images.

The deep learning model 108 is trained by, (i) extracting a first set of features from the historical satellite images, (ii) merging the first set of features to obtain feature maps, the feature maps include at least one of corners, or edges in the historical satellite images, (iii) extracting a second set of features by decreasing a size of each feature map independently, (iv) determining the set of historical hydrological parameters from the second set of parameters using a max-pooling method, (v) backpropagating a loss function to the trained deep learning model to optimize the set of historical hydrological parameters and the second set of features such that the loss function becomes zero, (vi) generating an optimized set of historical hydrological parameters as the historical hydrological parameters of the historical satellite images, and (vii) providing the historical hydrological parameters as the training data for training the deep learning model.

The soil moisture data generator module 212 generates using the trained deep learning model, high resolution volumetric soil moisture data of the geographical area on daily basis.

In some embodiments, the soil moisture data upscaling algorithm uses a vegetation-based lookup table to relate a microwave polarization index to soil moisture data estimates, a volumetric heat capacity of soil increases when a soil layer becomes wetter and a greater water content corresponds to a smaller temperature variation. The upscaling algorithm is used to stretch a lower resolution image to a larger display by copying pixels of a lower resolution image and filling out all pixels of the higher resolution display repeatedly.

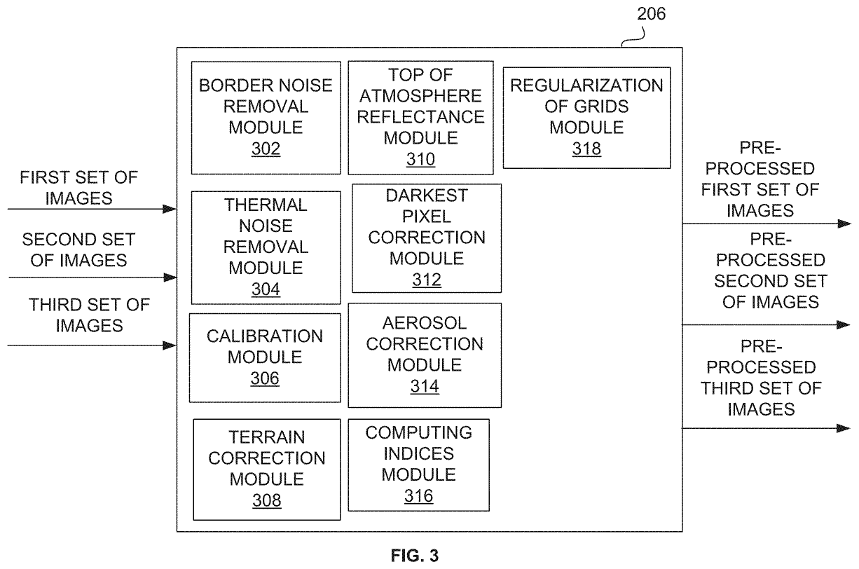

FIG. 3 illustrates a block diagram of a pre-processing module 206 according to some embodiments herein. The block diagram of the pre-processing module 206 includes a border noise removal module 302, a thermal noise removal module 304, a calibration module 306, a terrain correction module 308, a top of atmosphere reflectance module 310, a darkest pixel correction module 312, an aerosol correction module 314, a computing indices module 316, and a regularization of grids module 318. The border noise removal module 302 applies border noise removal on the first set of images to remove low-intensity noise and invalid data on the edges of the first set of images. The C-SAR instrument of the first satellite 102A provides an imaging capability to capture synthetic aperture radar (SAR) data at high arid medium resolutions for land, coastal zones and ice observations. The thermal noise removal module 304 removes thermal noise from the first set of images. The thermal noise removal reduces noise effects by normalizing the backscatter signal of the second set of images resulting in discontinuities among the second set of images, The operational land imager of Landsat-8 captures the second set of images in 9 spectral bands. The calibration module 306 calibrates the second set of images to convert the digital pixel values of the first set of images to radiometrically calibrated synthetic aperture radar (SAR) backscatter data, The terrain correction module 308 applies a terrain correction on SAR data. The SAR data includes distortions that are related to side geometry. The geometric distortions are compensated with terrain corrections such as foreshortening and shadow, using a digital elevation to correct the location of each pixel.

The top of atmosphere reflectance module 310 converts the second set of images to the top of atmosphere reflectance. The second set of images is not directly used to compute soil moisture data because the second set of images is a representation of spectral data without physical magnitude, thereby the second set of images is converted to top of reflectance. The algorithm considers the effects of vegetation cover on soil moisture data retrieval and introduces the vegetation fractional parameter (NDVI) into the algorithm, Therefore, the soil moisture data upscaling algorithm uses a vegetation-based lookup table to relate the microwave polarization index to soil moisture data estimates. The volumetric heat capacity of soil increases when the soil layer becomes wetter and the greater water content corresponds to a smaller temperature variation. The upscaling algorithm is used to stretch a lower resolution image to a larger display by copying pixels of a lower resolution image and filling out all pixels of the higher resolution display repeatedly.

The darkest pixel correction module 312 applies the darkest pixel correction on the second set of images, The darkest pixel may correspond to a large water body or other dark objects. The darkest pixel correction module 312 may utilize the darkest pixel correction method for applying the darkest pixel correction. The darkest pixel correction method may remove the effects of atmospheric scattering from the second set of images by subtracting a pixel value that represents a background signature from each band. The aerosol correction module 314 applies an aerosol correction to the second set of images. The aerosol correction module 314 may utilize the aerosol correction method. The aerosol correction method may identify dark pixels in the second set of images characterized by the very low top of canopy reflectance values for the shorter wavelengths where aerosol contribution to the reflectance at the top of the atmosphere is the largest. The aerosol correction is applied to eliminate atmospheric effects on the top of atmosphere reflectance. The computing indices module 316 computes NDVI and Land surface temperature from the second set of images. The multi-purpose microwave (MW) imager of the third satellite 102C may provide the third set of images once per day. The regularization of grids module 318 regularizes the third set of images into one or more grids. In some embodiments, the regularization of grids module 318 may divide the entire area into a number of one or more grids and are provided with outlines for processing. The first set of images is pre-processed to obtain the first pre-processed set of images. The second set of images is pre-processed to obtain a second pre-processed set of images. The third set of images is pre-processed to obtain a third pre-processed set of images.

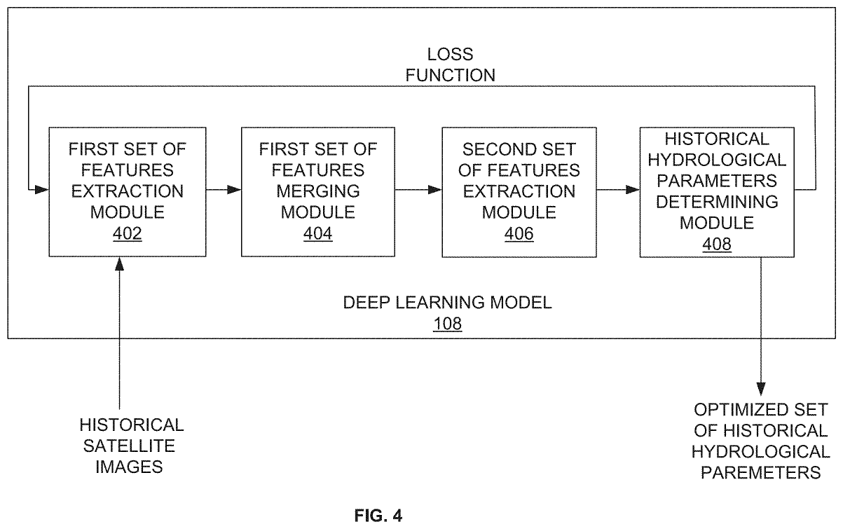

FIG. 4 illustrates a block diagram of a deep learning model 108 according to some embodiments herein. The deep learning model 108 includes a first set of features extraction module 402, a first set of features merging module 404, a second set of features extraction module 406, and a historical hydrological parameters determining module 408. The first set of features extraction module 402 extracts a first set of features from the historical satellite images. The first set of features merging module 404 merges the first set of features to obtain feature maps. In some embodiments, the feature maps include at least one of the corners or edges in the historical satellite images, The second set of features extraction module 406 extracts the second set of features by decreasing the size of each feature map independently. The historical hydrological parameters determining module 408 determine the set of historical hydrological parameters from the second set of parameters using a max-pooling method. The historical hydrological parameters determining module 408 backpropagates a loss function to the trained deep learning model to optimize the set of historical hydrological parameters and the second set of features such that the loss function becomes zero. The historical hydrological parameters determining module 408 generate an optimized set of historical hydrological parameters as the historical hydrological parameters of the historical satellite images. The historical hydrological parameters are provided as the training data for training the deep learning model 108.

In some embodiments, the feature maps are generated using a convolution filter to the set of historical satellite images.

In some embodiments, a maximum element of a region of each feature map is selected to determine the set of historical hydrological parameters from the second set of parameters using the max pooling method. The max pooling method calculates the maximum or largest value in each pixel of each feature map, thereby extracting low-level features like edges, points, etc. Hence, the extraction of smooth and sharp features is done by reducing variance and computations, and thereby computational cost gets reduced.

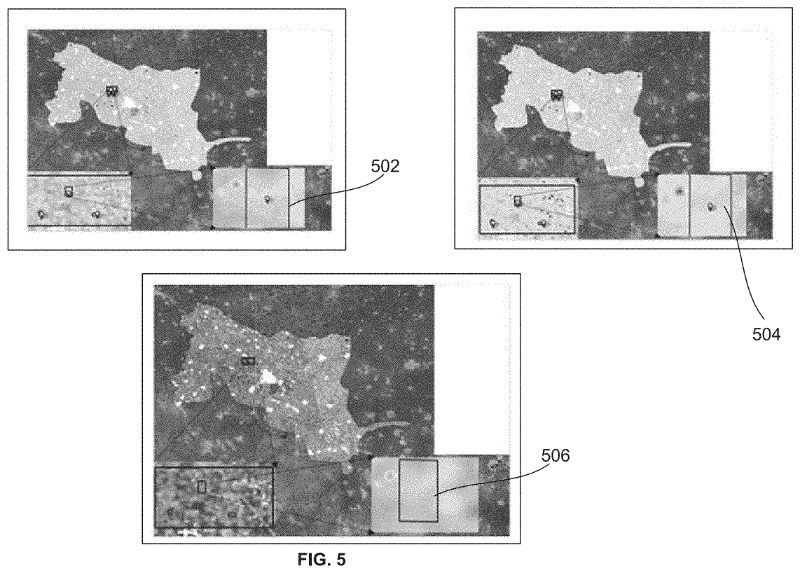

FIG. 5 illustrates an exemplary view of spatial variation of soil moisture data according to some embodiments herein. The exemplary view of spatial variation of soil moisture data includes soil moisture data at the start of the season is shown at 502. The exemplary view of spatial variation of soil moisture data includes soil moisture data at the vegetation stage of the season is shown at 504. The exemplary view of spatial variation of soil moisture data includes soil moisture data at end of harvest season is shown at 506. A 35-40% of volumetric soil moisture data at a resolution of 20 meters at the start of the season is shown at 502. A 50-55% of volumetric soil moisture data at a resolution of 20 meters at the vegetation stage of the season is shown at 504. A 60-65% of volumetric soil moisture data at a resolution of 20 meters at the start of the season is shown at 506.

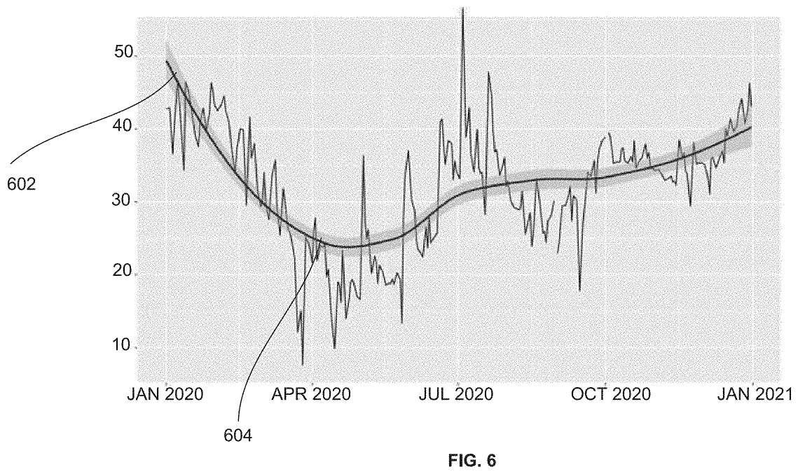

FIG. 6 illustrates a graphical representation of the temporal plot of soil moisture data according to some embodiments herein. The temporal plot of soil moisture data depicts volumetric soil moisture data on Y-axis and time on X-axis. The plot depicts a more percentage of volumetric soil moisture data in January 2020 at 602 which is the harvest season of the year. The plot depicts a low percentage of volumetric soil moisture data in April 2020 at 604 which is the end season of the year.

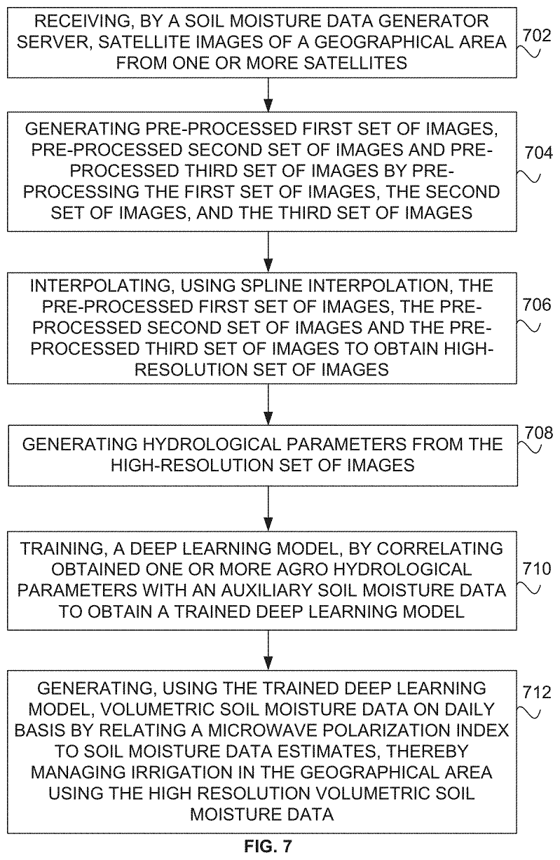

FIG. 7 is a flow diagram of a method for generating soil moisture data from satellite images of a geographical area using a deep learning model 108 according to some embodiments herein. At step 702, the method includes, receiving, by a soil moisture data generator server, satellite images of the geographical area from satellites. In some embodiments, the satellite images include a first set of satellite images that are captured in a first set of spectral bands by a first satellite, a second set of satellite images that are captured in a second set of spectral hands by a second satellite and a third set of satellite images that are captured in a third set of spectral hands by a third satellite. At step 704, the method includes, generating a pre-processed first set of images, a pre-processed second set of images, and a pre-processed third set of images by pre-processing the first set of satellite images, the second set of satellite images, and the third set of satellite images. At a step of 706, the method includes, interpolating the pre-processed first set of images, the pre-processed second set of images, and the pre-processed third set of images to generate a high-resolution set of images, the high-resolution set of images are generated by increasing a temporal resolution of the pre-processed first set of images, the pre-processed second set of images, and the pre-processed third set of images to a daily scale using a spline interpolation. At a step of 708, the method includes generating hydrological parameters from the high-resolution set of images. At 710, the method includes, training a deep learning model by providing historical hydrological parameters and historical soil moisture data associated with historical satellite images as training data to generate a trained deep learning model. At 712, the method includes, generating, using the trained deep learning model, high resolution volumetric soil moisture data of the geographical area on daily basis by relating a microwave polarization index to soil moisture data estimates, thereby enabling the irrigation management for the geographical area using the high resolution volumetric soil moisture data.

In some embodiments, the method includes pre-processing of the first set of satellite images, and the second set of satellite images by, (i) applying atmospheric corrections to remove effects of atmosphere on the first set of satellite images to obtain the atmospherically corrected first set of satellite images and atmospherically corrected second set of satellite images, (ii) applying a border noise removal on the atmospherically corrected first set of images to remove low-intensity noise and invalid data on edges of the atmospherically corrected first set of images, (iii) calibrating the atmospherically corrected second set of satellite images to convert digital pixel values of the atmospherically corrected first set of images to radiometrically calibrated synthetic aperture radar (SAR) backscatter data, (iv) applying a terrain correction on the SAR backscatter data by removing distortions that are related to side geometry, and (v) converting the SAR backscatter data into the top of atmosphere reflectance.

In some embodiments, the method includes training the deep learning model by, (i) extracting a first set of features from the historical satellite images, (ii) merging the first set of features to obtain feature maps, the feature maps include at least one of corners, or edges in the historical satellite images, (iii) extracting a second set of features by decreasing a size of each feature map independently, (iv) determining the set of historical hydrological parameters from the second set of parameters using a max-pooling method, (v) backpropagating a loss function to the trained deep learning model to optimize the set of historical hydrological parameters and the second set of features such that the loss function becomes zero, (vi) generating an optimized set of historical hydrological parameters as the historical hydrological parameters of the historical satellite images, and (vii) providing the historical hydrological parameters as the training data for training the deep learning model.

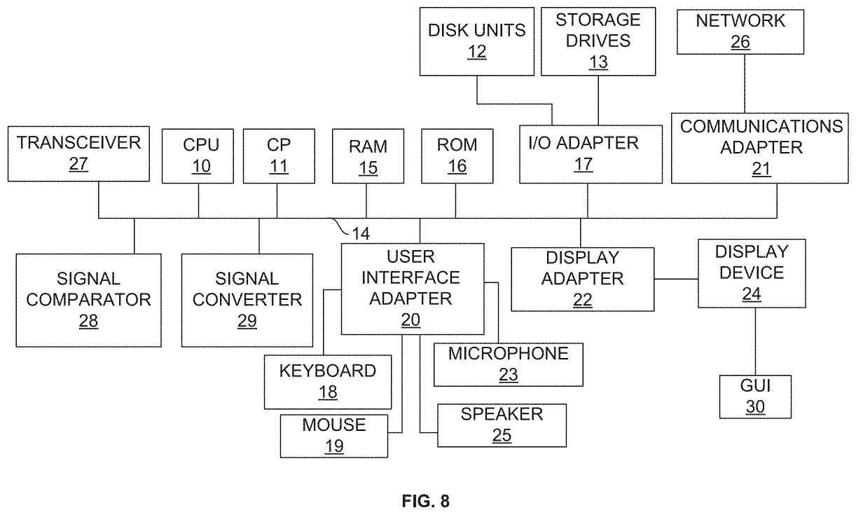

A representative hardware environment for practicing the embodiments herein is depicted in FIG. 8, with reference to FIGS. 1 through 7. This schematic drawing illustrates a hardware configuration of a soil moisture data generator server 106/computer system/computing device in accordance with the embodiments herein. The system includes at least one processing device CPU 10 and at least one graphical processing device GPU 38 that may be interconnected via system bus 14 to various devices such as a random access memory (RAM) 12, read-only memory (ROM) 16, and an input/output (I/O) adapter 18. The I/O adapter 18 can connect to peripheral devices, such as disk units 38 and program storage devices 40 that are readable by the system. The system can read the inventive instructions on the program storage devices 40 and follow these instructions to execute the methodology of the embodiments herein. The system further includes a user interface adapter 22 that connects a keyboard 28, mouse 30, speaker 32, microphone 34, and/or other user interface devices such as a touch screen device (not shown) to the bus 14 to gather user input. Additionally, a communication adapter 20 connects the bus 14 to a data processing network 42, and a display adapter 24 connects the bus 14 to a display device 26, which provides a graphical user interface (GUI) 36 of the output data in accordance with the embodiments herein, or which may be embodied as an output device such as a monitor, printer, or transmitter, for example.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope.

What is claimed is:

1. A system for generating soil moisture data from a plurality of satellite images of a geographical area using a deep learning model for managing irrigation in the geographical area, wherein the system comprises:
a plurality of satellites that captures a plurality of satellite images of the geographical area, wherein the plurality of satellite images comprises a set of spectral bands;
a soil moisture data generator server that receives the plurality of satellite images of the geographical area from the plurality of satellites, wherein the plurality of satellite images comprises a first set of satellite images that are captured in a first set of spectral bands by a first satellite, a second set of satellite images that are captured in a second set of spectral bands by a second satellite and a third set of satellite images that are captured in a third set of spectral bands by a third satellite, wherein the soil moisture data generator server comprises,
a memory that stores a database and a set of modules;
a processor in communication with the memory, the processor is configured to:
generate a pre-processed first set of images, a pre-processed second set of images, and a pre-processed third set of images by pre-processing the first set of satellite images, the second set of satellite images, and the third set of satellite images;
characterized in that,
interpolate the pre-processed first set of images, the pre-processed second set of images, and the pre-processed third set of images to generate a high-resolution set of images, wherein the high-resolution set of images are generated by increasing a temporal resolution of the pre-processed first set of images, the pre-processed second set of images, and the pre-processed third set of images to a daily scale using a spline interpolation;
generate a plurality of hydrological parameters from the high-resolution set of images;
train a deep learning model by providing a plurality of historical hydrological parameters and a plurality of historical soil moisture data associated with a plurality of historical satellite images as training data to generate a trained deep learning model; and
generate, using the trained deep learning model, high resolution volumetric soil moisture data of the geographical area on daily basis by relating a microwave polarization index to soil moisture data estimates, thereby managing the irrigation in the geographical area using the high resolution volumetric soil moisture data.

2. The system of claim 1, wherein the processor is configured to pre-process the first set of satellite images, and the second set of satellite images by,
applying atmospheric corrections to remove effects of atmosphere on the first set of satellite images to obtain the atmospherically corrected first set of satellite images and the atmospherically corrected second set of satellite images;
applying a border noise removal on the atmospherically corrected first set of images to remove low-intensity noise and invalid data on edges of the atmospherically corrected first set of images;
calibrating the atmospherically corrected second set of satellite images to convert digital pixel values of the atmospherically corrected first set of images into radiometrically calibrated synthetic aperture radar (SAW) backscatter data;
applying a terrain correction on the SAR backscatter data by removing distortions that are related to a side geometry; and
converting the SAR backscatter data into the top of atmosphere reflectance.

3. The system of claim 1, wherein the processor is configured to apply a darkest pixel correction that corresponds to a large water body to the second set of satellite images, wherein the processor is configured to divide the geographical area into a plurality of grids by regularizing the third set of satellite images, wherein the processor is configured to apply an aerosol correction to the second set of images to eliminate atmospheric effects on the top of atmosphere reflectance, wherein the processor is configured to compute a normalized difference vegetation index (NDVI) and land surface temperature from the second set of satellite images.

4. The system of claim 1, wherein the processor is configured to train the deep learning model by,
extracting a first set of features from the plurality of historical satellite images;
merging the first set of features to obtain a plurality of feature maps, wherein the plurality of feature maps comprises at least one of corners, or edges in the plurality of historical satellite images;
extracting a second set of features by decreasing a size of each feature map independently;
determining the set of historical hydrological parameters from the second set of features using a max-pooling method;
backpropagating a loss function to the deep learning model to optimize the set of historical hydrological parameters and the second set of features such that the loss function becomes zero;
generating an optimized set of historical hydrological parameters as the historical hydrological parameters of the plurality of historical satellite images; and
providing the historical hydrological parameters as the training data for training the deep learning model.

5. The system of claim 4, wherein the plurality of feature maps are generated using a convolution filter to the plurality of historical satellite images.

6. The system of claim 4, wherein a maximum element of a region of each feature map is selected to determine the set of historical hydrological parameters from the second set of features using the max pooling method.

7. The system of claim 1, wherein the processor is configured to analyze a vegetation-based lookup table to relate the microwave polarization index to the soil moisture data estimates using a soil moisture data upscaling algorithm, wherein a volumetric heat capacity of soil increases when a soil layer becomes wetter and a greater water content corresponds to a smaller temperature variation.

8. A processor-implemented method for generating soil moisture data from a plurality of satellite images of a geographical area using a deep learning model for managing irrigation in the geographical area, wherein the method comprises:
receiving, by a soil moisture data generator server, a plurality of satellite images of the geographical area from a plurality of satellites, wherein the plurality of satellite images comprises a first set of satellite images that are captured in a first set of spectral bands by a first satellite, a second set of satellite images that are captured in a second set of spectral bands by a second satellite and a third set of satellite images that are captured in a third set of spectral bands by a third satellite;
generating a pre-processed first set of images, a pre-processed second set of images, and a pre-processed third set of images by pre-processing the first set of satellite images, the second set of satellite images, and the third set of satellite images;
interpolating the pre-processed first set of images, the pre-processed second set of images, and the pre-processed third set of images to generate a high-resolution set of images, wherein the high-resolution set of images are generated by increasing a temporal resolution of the pre-processed first set of images, the pre-processed second set of images, and the pre-processed third set of images to a daily scale using a spline interpolation,
characterized in that,
generating a plurality of hydrological parameters from the high-resolution set of images;
training a deep learning model by providing a plurality of historical hydrological parameters and a plurality of historical soil moisture data associated with a plurality of historical satellite images as training data to generate a trained deep learning model; and
generating, using the trained deep learning model, high resolution volumetric soil moisture data of the geographical area on daily basis by relating a microwave polarization index to soil moisture data estimates, thereby managing the irrigation in the geographical area using the high resolution volumetric soil moisture data.

9. One or more non-transitory computer-readable storage medium storing. the one or more sequence of instructions, which when executed by the one or more processors, causes to perform a method of generating soil moisture data from a plurality of satellite images of a geographical area using a deep learning model for managing irrigation in the geographical area, wherein the method comprises:
receiving, by a soil moisture data generator server, a plurality of satellite images of the geographical area from a plurality of satellites, wherein the plurality of satellite images comprises a first set of satellite images that are captured in a first set of spectral bands by a first satellite, a second set of satellite images that are captured in a second set of spectral bands by a second satellite and a third set of satellite images that are captured in a third set of spectral bands by a third satellite;
generating a pre-processed first set of images, a pre-processed second set of images, and a pre-processed third set of images by pre-processing the first set of satellite images, the second set of satellite images, and the third set of satellite images;
characterized in that,
interpolating the pre-processed first set of images, the pre-processed second set of images, and the pre-processed third set of images to generate a high-resolution set of images, wherein the high-resolution set of images are generated by increasing a temporal resolution of the pre-processed first set of images, the pre-processed second set of images, and the pre-processed third set of images to a daily scale using a spline interpolation;
generating a plurality of hydrological parameters from the high-resolution set of images;
training a deep learning model by providing a plurality of historical hydrological parameters and a plurality of historical soil moisture data associated with a plurality of historical satellite images as training data to generate a trained deep learning model; and
generating, using the trained deep learning model, high resolution volumetric soil moisture data of the geographical area on daily basis by relating a microwave polarization index to soil moisture data estimates, thereby managing the irrigation in the geographical area using the high resolution volumetric soil moisture data.

10. The processor-implemented method of claim 8, wherein the method comprises pre-processing the first set of satellite images, and the second set of satellite images by,
applying atmospheric corrections to remove effects of atmosphere on the first set of satellite images to obtain the atmospherically corrected first set of satellite images and the atmospherically corrected second set of satellite images;
applying a border noise removal on the atmospherically corrected first set of images to remove low-intensity noise and invalid data on edges of the atmospherically corrected first set of images;
calibrating the atmospherically corrected second set of satellite images to convert digital pixel values of the atmospherically corrected first set of images into radiometrically calibrated synthetic aperture radar (SAR) backscatter data;
applying a terrain correction on the SAR backscatter data by removing distortions that are related to a side geometry; and
converting the SAR data into the top of atmosphere reflectance.

11. The processor-implemented method of claim 8, wherein the method comprises train the deep learning model by,
extracting a first set of features from the plurality of historical satellite images;
merging the first set of features to obtain a plurality of feature maps, wherein the plurality of feature maps comprises at least one of corners, or edges in the plurality of historical satellite images;
extracting a second set of features by decreasing a size of each feature map independently;
determining the set of historical hydrological parameters from the second set of features using a max-pooling method;
backpropagating a loss function to the deep learning model to optimize the se of historical hydrological parameters and the second set of features such that the loss function becomes zero;
generating an optimized set of historical hydrological parameters as the historical hydrological parameters of the plurality of historical satellite images; and
providing the historical hydrological parameters as the training data for training the deep learning model.

12. The processor-implemented method of claim 8, wherein the method further comprises applying a darkest pixel correction that corresponds to a large water body to the second set of satellite images, wherein the method further comprises dividing the geographical area into a plurality of grids by regularizing the third set of satellite images, wherein the method further comprises applying an aerosol correction to the second set of images to eliminate atmospheric effects on the top of atmosphere reflectance, wherein the method further comprises computing a normalized difference vegetation index (NDVI) and land surface temperature from the second set of satellite images.

13. The processor-implemented method of claim 12, wherein the plurality of feature maps are generated using a convolution filter to the plurality of historical satellite images.

14. The processor-implemented method of claim 12, wherein a maximum element of a region of each feature map is selected to determine the set of historical hydrological parameters from the second set of features using the max pooling method.

15. The processor-implemented method of claim 9, wherein the method further comprises analyzing a vegetation-based lookup table to relate the microwave polarization index to the soil moisture data estimates using a soil moisture data upscaling algorithm, wherein a volumetric heat capacity of soil increases when a soil layer becomes wetter and a greater water content corresponds to a smaller temperature variation.
